(19) United States
(12) Patent Application Publication
(10) Pub. No.: US 2020/0308528 A1
(43) Pub. Date: Oct. 1, 2020

(54) APPARATUS AND METHOD FOR HUMIDIFYING CELL CULTURES

(71) Applicant: Xona Microfluidics, Inc., Research Triangle Park, NC (US)

(72) Inventor: Anne Marion Taylor, Carrboro, NC (US)

(21) Appl. No.: 16/829,699

(22) Filed: Mar. 25, 2020

Related U.S. Application Data
(60) Provisional application No. 62/824,050, filed on Mar. 26, 2019.
(60) Provisional application No. 62/981,871, filed on Feb. 26, 2020.

(51) Int. Cl. C12M 1/34; F24F 6/00; C12M 1/12

(52) U.S. Cl. CPC C12M 41/32; C12M 25/02; F24F 6/00

(57) ABSTRACT

A device for humidifying culturing cells growing on a substrate comprises a first body member having an inner surface defining a pair of spaced troughs opening to the inner surface for holding a humidifying fluid. A portion of the inner surface between the troughs defines at least one recess having an opening for receiving the substrate. A second body member has an inner surface. The second body member is configured to be mounted to the first member such that the substrate is disposed between the inner surface of the first body member and the inner surface of the second body member.

### CROSS-REFERENCE TO RELATED APPLICATIONS

This application claims the benefit of U.S. Provisional Application No. 62/824,050, filed Mar. 26, 2019, and U.S. Provisional Application No. 62/981,871, filed Feb. 26, 2020, the contents of both of which are incorporated by reference herein in their entirety.

### BACKGROUND

An apparatus and method for growing cells is described and, more particularly, an apparatus and method for humidifying cells during cell culturing using a humidifying tray with a microfluidic culture device.

Standard open culture methods are conventional for most cell types. However, some cell types, such as neurons, can be asymmetrical and parts of the cell may be specialized. Neurons represent a cell type cultured for neuroscience research, toxicity testing and drug screening. Neurons are polarized and have many processes, such as axons, that extend over relatively long distances and benefit from a culturing method providing selective isolation and treatment.

U.S. Pat. No. 7,419,822, the contents of which are hereby incorporated by reference in their entirety, describes a neuron culture device that combines microfabrication, microfluidic, and/or surface micropatterning techniques to create a neuronal culturing device that enables selective neuritic isolation and treatment. The microfabricated neuronal device has two or more compartments interconnected by a region having micron-sized grooves, or microchannels or micro-grooves, at the bottom of a physical barrier. A researcher plates neuronal cells into a first somal, or cell body, compartment. The configuration of the microfluidic device enables neurites, or other portion of a cellular organism, to then grow across the barrier via the grooves and extend into a second neuritic compartment. The size of the grooves is designed to limit the neurons to the somal compartment, or chamber, while allowing the growing neuritic processes to cross from one chamber to another. Well-defined grooves with controlled dimension thus allow each chamber to function in a fluidically isolated manner. A user may selectively apply positive or negative stimuli to distal portions of the neurites.

The present Applicant makes and sells neuron culturing devices as described above.

Devices are sold in two different materials: poly(dimethylsiloxane) (PDMS) and cyclic olefin copolymer (COC). The culturing device fabricated in PDMS is sold as silicone devices. Silicone devices are placed onto an optically transparent substrate (e.g., coverglass) by the end user making them optically transparent for microscopy applications. The culturing device fabricated in COC is sold under the name XonaChips®. XonaChips® are pre-assembled to a substrate substantially the same size as a conventional microscope slide and are optically transparent for microscopy applications.

One factor important for culturing neurons when using silicone devices or XonaChips® is the humidity of the culture environment. Maintaining a high humidity environment is critical to viability of the neurons in the device.

For the foregoing reasons, there is a need for an apparatus and method for humidifying neuronal cells during culturing. Ideally, the new apparatus and method should work well with current microfluidic cell culture devices, including multi-compartment culturing devices for growing nerve cells for use in neuroscience research.

### SUMMARY

A device is described for use in culturing cells. The cell culturing device comprises a first body member having an inner surface defining a pair of spaced troughs opening to the inner surface for holding humidifying fluid. A portion of the inner surface between the troughs defining at least one recess having an opening. A second body member has an inner surface and a substrate is provided for cell growth. The substrate is configured to be received in the recess overlying the opening in the first body member. The second body member is configured to be mounted to the first member such that the substrate is disposed between the inner surface of the first body member and the inner surface of the second body member.

A device for humidifying culturing cells growing on a substrate is also described. The cell humidifying device comprises a first body member having an inner surface defining a pair of spaced troughs opening to the inner surface for holding a humidifying fluid. A portion of the inner surface between the troughs defines at least one recess having an opening for receiving the substrate. A second body member has an inner surface, wherein the second body member is configured to be mounted to the first member such that the substrate is disposed between the inner surface of the first body member and the inner surface of the second body member.

In one aspect, the first body member is elongated, and the troughs in the first body member are disposed at opposite ends of the first body member.

In another aspect, the first body member and the second body member may be transparent or opaque.

In a further aspect, the portion of the first body member defining the recess has a continuous ledge for receiving and supporting the substrate.

In one embodiment, each of the first body member and the second body member comprises a base portion terminating in longitudinal edges, and a continuous peripheral wall extending from the base portion and terminating in longitudinal edges. The base portion and peripheral wall of the second body member define an open cavity configured to receive the peripheral wall of the first body member for connecting the first body member and the second body member.

In one aspect of this embodiment, a flange extends along at least a portion of the longitudinal edges of the base portion of the first body member, and the terminal edges of the peripheral wall of the second body member contact the flange of the first body member when connecting for spacing the inner surface of the first body member from the inner surface of the second body member. The distance from the base portion to terminal edges of the wall of the first body member may be less than the distance from the base portion to the terminal edges of the wall of the second body member.

Another embodiment of a device for use in culturing cells is described. The cell culturing device comprises a first body member having an inner surface and an outer surface, the inner surface defining a peripheral trough opening to the inner surface for holding humidifying fluid, the outer surface defining at least one recess within the area defined by the trough, and having at least one opening between the inner surface and the recess. A substrate having a surface for cell growth is configured to be received in the recess overlying the at least one opening in the first body member such that the surface for cell growth is disposed adjacent to the outer surface of the first body member. A second body member having an inner surface is configured to be mounted to the first member.

In one embodiment, the first body member is elongated, and the trough is continuous around the periphery of the first body member.

In another aspect, the portion of the first body member defining the recess has a continuous ledge for receiving and supporting the substrate.

In one embodiment, each of the first body member and the second body member comprises a base portion terminating in longitudinal edges, and a continuous peripheral wall extending from the base portion and terminating in longitudinal edges. The base portion and peripheral wall of the second body member define an open cavity configured to receive the peripheral wall of the first body member for connecting the first body member and the second body member.

In one aspect of this embodiment, a flange extends along at least a portion of the longitudinal edges of the base portion of the first body member, and the terminal edges of the peripheral wall of the second body member contact the flange of the first body member when connecting for spacing the inner surface of the first body member from the inner surface of the second body member. The distance from the base portion to terminal edges of the wall of the first body member may be less than the distance from the base portion to the terminal edges of the wall of the second body member.

### BRIEF DESCRIPTION OF THE DRAWINGS

For a more complete understanding of an apparatus and method for humidifying culture cells, reference should now be had to the embodiments shown in the accompanying drawings and described below. In the drawings:

FIG. 1 is a top exploded perspective view of an embodiment of a humidifier tray assembly for use in culturing cells.

FIG. 2 is a top plan view of the bottom tray of the humidifier tray assembly as shown in FIG. 1.

FIG. 3 is a side elevation view of the bottom tray as shown in FIG. 2.

FIG. 4 is a transverse cross-section view of the bottom tray and taken along line 4-4 of FIG. 2.

FIG. 5 is a top perspective of the bottom tray as shown in FIG. 2 holding two Xonachips.

FIG. 6 is a top plan view of the bottom tray holding two Xonachips as shown in FIG. 5.

FIG. 7 is a top perspective view of an embodiment of a microfabricated device configured for cell culture.

FIG. 8 is a bottom perspective view of the microfabricated device as shown in FIG. 7.

FIG. 9 is a top plan view of the microfabricated device as shown in FIG. 7.

FIG. 10 is a bottom plan view of the microfabricated device as shown in FIG. 7.

FIG. 11 is a top plan view of the microfabricated device as shown in FIG. 3 with features of the device shown in phantom.

FIG. 12 is a side elevation view of the microfabricated device as shown in FIG. 1.

FIG. 13 is a longitudinal cross-section view of the microfabricated device taken along line 13-13 of FIG. 12.

FIG. 14 is a longitudinal cross-section view of the microfabricated device taken along line 14-14 of FIG. 12.

FIG. 15 is a transverse cross-section view of the microfabricated device taken along line 15-15 of FIG. 12.

FIG. 16 is an up close perspective view of the microfabricated device as shown in FIG. 8.

FIG. 17 is an up close perspective view of the microfabricated device as shown in FIG. 16.

FIG. 18 is a top perspective view of another embodiment of an apparatus for humidifying culture cells.

FIG. 19 is a bottom perspective view of the humidifying apparatus as shown in FIG. 18.

FIG. 20 is a top plan view of the humidifying apparatus as shown in FIG. 18 with features of the apparatus shown in phantom.

FIG. 21 is a bottom plan view of the humidifying apparatus as shown in FIG. 18.

FIG. 22 is a side elevation view of the humidifying apparatus as shown in FIG. 18.

FIG. 23 is a transverse cross-section view of the humidifying apparatus taken along line 23-23 of FIG. 20.

FIG. 24 is a longitudinal cross-section view of the humidifying apparatus taken along line 24-24 of FIG. 20.

FIG. 25 is an up close elevation view of the humidifying apparatus as shown in FIG. 24.

FIG. 26 is a bottom plan view of the humidifying apparatus as shown in FIG. 21 including microfabricated devices.

### DESCRIPTION

Certain terminology is used herein for convenience only and is not to be taken as a limiting. For example, words such as “upper,” “lower,” “left,” “right,” “horizontal,” “vertical,” “upward,” “downward,” “top” and “bottom” merely describe the configurations shown in the Figures. Indeed, the components may be oriented in any direction and the terminology, therefore, should be understood as encompassing such variations unless specified otherwise. The words “interior” and “exterior” refer to directions toward and away from, respectively, the geometric center of the core and designated parts thereof. The terminology includes the words specifically mentioned above, derivatives thereof and words of similar import.

As used herein, “axial” is deemed to mean parallel to an axis of an apparatus or device, but not necessarily coaxial therewith.

As used herein, “cell” means any cell or cells, as well as viruses or any other particles having a microscopic size, e.g. a size that is similar to that of a biological cell, and includes any prokaryotic or eukaryotic cell, e.g., bacteria, fungi, plant and animal cells. Cells are typically spherical, but can also be elongated, flattened, deformable and asymmetrical, i.e., non-spherical. The size or diameter of a cell typically ranges from about 0.1 to 120 microns, and typically is from about 1 to 50 microns. A cell may be living or dead. Since the apparatus is directed to humidifying materials having a size similar to a biological cell (e.g. about 0.1 to 120 microns) any material having a size similar to a biological cell can be humidified using the humidifying apparatus.

An apparatus and a method for humidifying cells during culturing is described. The humidifying apparatus and method may be implemented in a lab setting or in a commercial environment.

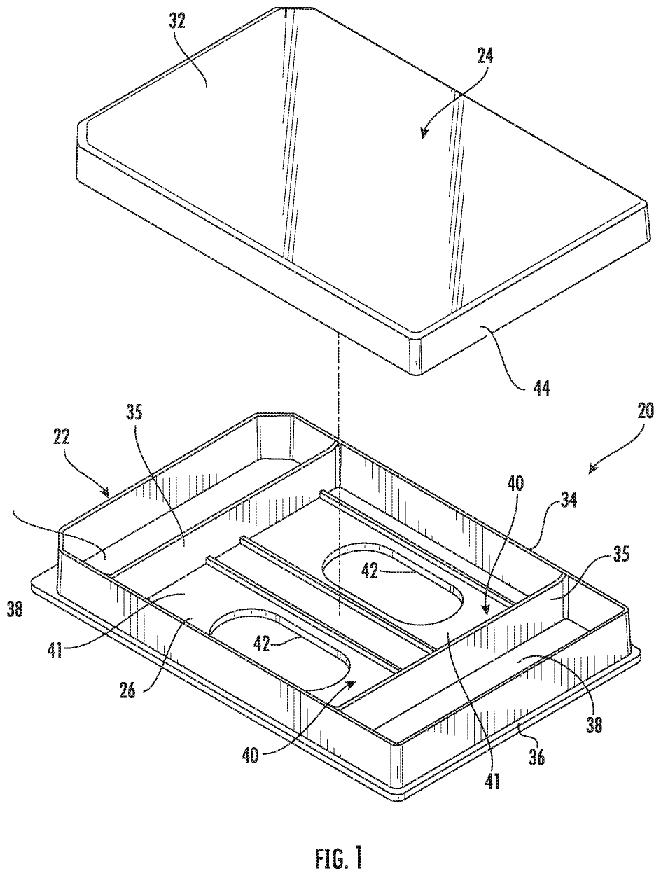

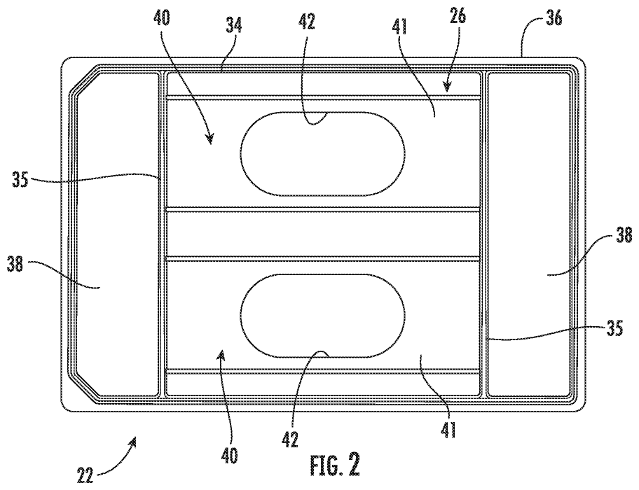

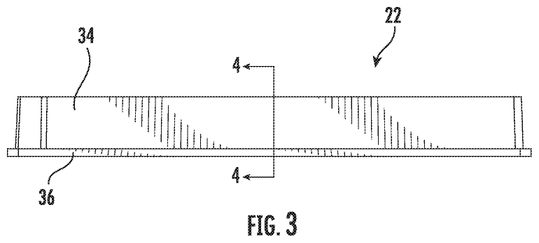

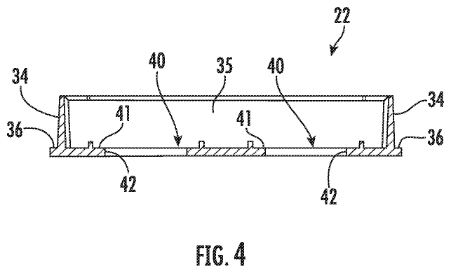

In one application, the apparatus and method for humidifying cells utilize microfluidic devices to provide humidification to cell types growing on the devices. Referring now to the drawings, wherein like reference numerals indicate the same or similar elements throughout the several views, an embodiment of an apparatus for humidifying cells during culturing is shown. As shown in FIG. 1, the humidifying apparatus comprises a tray assembly which is generally designated at 20. The tray assembly 20 provides a closed environment for cell growth, as well as a means for easy loading of microfluidic devices into the tray assembly 20. The tray assembly 20 includes a bottom tray 22 and a cover 24, each of which has an inner surface 26, 28 and an outer surface 30, 32. In the embodiment shown in the drawings, the tray 22 and cover 24 are shown as generally rectangular, although it is understood that the tray assembly 20 is not so limited and may have any convenient shape in addition to rectangular.

The tray assembly 20 may be formed from a number of suitable materials, including plastics or glass. A tray assembly 20 formed of plastic can be bendable and resilient to a certain extent, and thus insures that the bottom tray 22 and the cover 24 can be readily assembled and separated from one another. In one embodiment, the tray assembly 20 is made of thermoformed, extruded or molded plastic resins. The tray assembly 20 may be substantially transparent or translucent for allowing observation of the interior of the tray assembly 20, including cellular material during growth. Alternatively, the tray assembly 20 may be opaque, such as black plastic, to prevent phototoxicity. It is understood that the tray assembly 20 is not intended to be limited by the materials listed here, but may be carried out using any material which allows the construction and operation of the humidifying apparatus described herein.

A continuous peripheral wall 34 extends orthogonally from the inner surface 26 of the bottom tray 22 and terminates in longitudinal edges. The wall 34 is spaced inwardly from the edges of the bottom tray 22 thereby forming an integral lip 36 extending along the edges of the tray. A transverse wall 35 interconnecting the peripheral wall and spaced from each end of the bottom tray 22 defines troughs 38 along the ends of the tray 20. The troughs 38 open onto the inner surface 26 of the bottom tray 22.

The inner surface 26 of the bottom tray 22 between the troughs 38 further defines two parallel rectangular recesses 40. The portions of the inner surface 26 of the bottom tray 22 defining the recesses include a continuous ledge 41. In one embodiment, the ledge 41 is configured to correspond to the size of a conventional glass or plastic microscope slide which may serve as a substrate for cell growth. Each of the recesses 40 may receive and support on the ledge 41 a microscope slide within the inner surface 26 of the bottom tray 22. The ledges 41 each have an oval window 42 opening into the bottom tray 22 such that at least a portion of the substrate can be optically observed through the window 42 in the bottom tray 22.

Referring now to the cover 24, the cover 24 includes a continuous peripheral wall 44 extending orthogonally from the inner surface 28 of the cover and terminating in longitudinal edges. The inner surface 28 and the wall 44 of the cover 24 define an open cavity for receiving the bottom tray 22 in an assembled condition. When assembled, the wall 44 of the cover 24 closely surrounds the wall 34 of the tray 22 for a slip-fit connection between the cover 24 and the bottom tray 22. In one embodiment, the distance of the terminal edge of the wall 44 of the cover 24 from the inner surface 28 of the cover is greater than the distance of the terminal edge of the wall 34 of the bottom tray 22 from the inner surface 26 of the tray. Accordingly, when the tray assembly 20 is the assembled condition, the terminal edges of the wall 44 of the cover 24 engage the lip 36 at the edge of the tray 22. This arrangement allows the user to predetermine the spacing between the inner surface 26 of the bottom tray 22 and the inner surface 28 of the cover 24.

In use, water or other fluid is added to the troughs 38 at each end of the tray assembly 20.

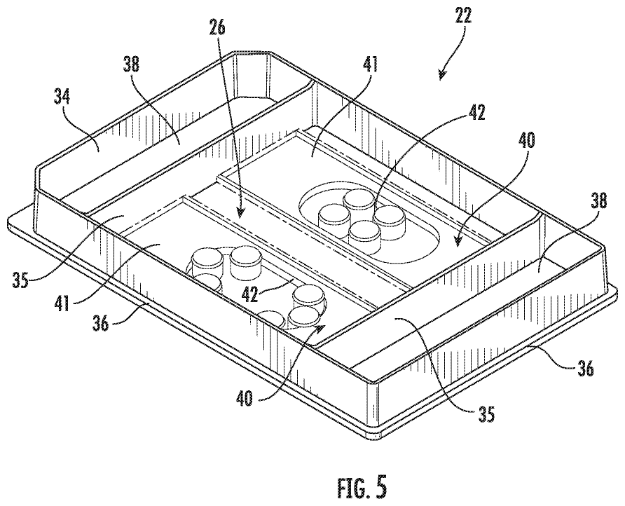

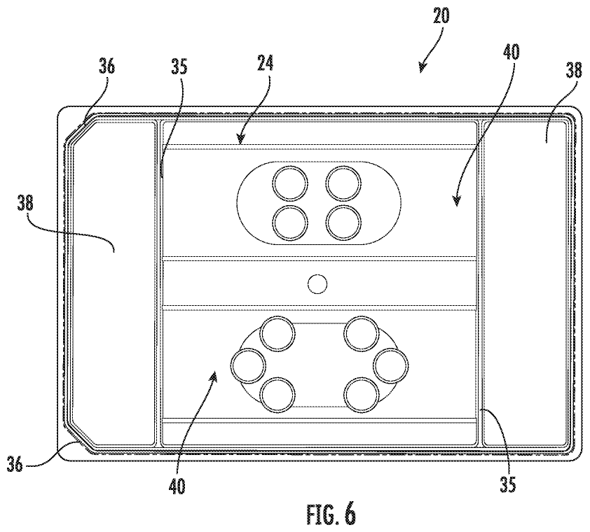

A flange parallel to the inner surface 26 may extend from the peripheral wall 34 or the transverse wall 35 partially over the troughs 38 to minimize loss of fluid during handling of the tray assembly 20. Cells are plated on the surfaces of a substrate configured to fit into the recesses 40 in the bottom tray 22. The substrates are then placed on the ledges 41 in the recesses 40 such that substrates overlie the windows 42 defined by the ledges 41. Referring to FIGS. 5 and 6, Xonachips are positioned in the recesses 40. The cover 24 is then held over the bottom tray 22 so that the wall 44 of the cover 24 is adjacent the wall 34 of the bottom tray 22. The wall 44 of the cover 24 define an area slightly larger than the area defined by the wall 34 of the bottom tray 22, which allows the cover 24 to slide onto the bottom tray 22 with a minimum of manual urging. During assembly as the tray assembly 20 slides together, the terminal edges of the wall 44 of the cover 24 ultimately engage the lip 36 around the periphery of the bottom tray 22. With the tray assembly 20 in the assembled condition, the troughs 38 and recesses 40 and supported substrates are enclosed between the inner surfaces 26, 28 of the bottom tray 22 and the cover 24.

In one embodiment, the tray assembly 20 may be configured such that it will fit on a microscope stage. An inverted microscope (not shown) may be used, with the objective lens below the tray assembly 20 and a light source above. The clear or translucent construction of the tray assembly 20 and the windows 42 in the bottom tray 22 allow the user to easily visualize the condition of the cells cultured on the substrates during cell proliferation.

To disassemble the tray assembly 20, the cover 24 is manually pulled away from the bottom tray 22 a sufficient distance to slidingly disengage the walls 34, 44.

It is understood that although the embodiments of the tray assembly 20 shown herein depict a close-fitting walled arrangement, other arrangements for joining the cover 24 and the bottom tray 22 are contemplated. For example, the cover 24 may be pivotally mounted to the bottom tray 22 via a hinge assembly along one side for pivotal movement between a first open position and a second closed position.

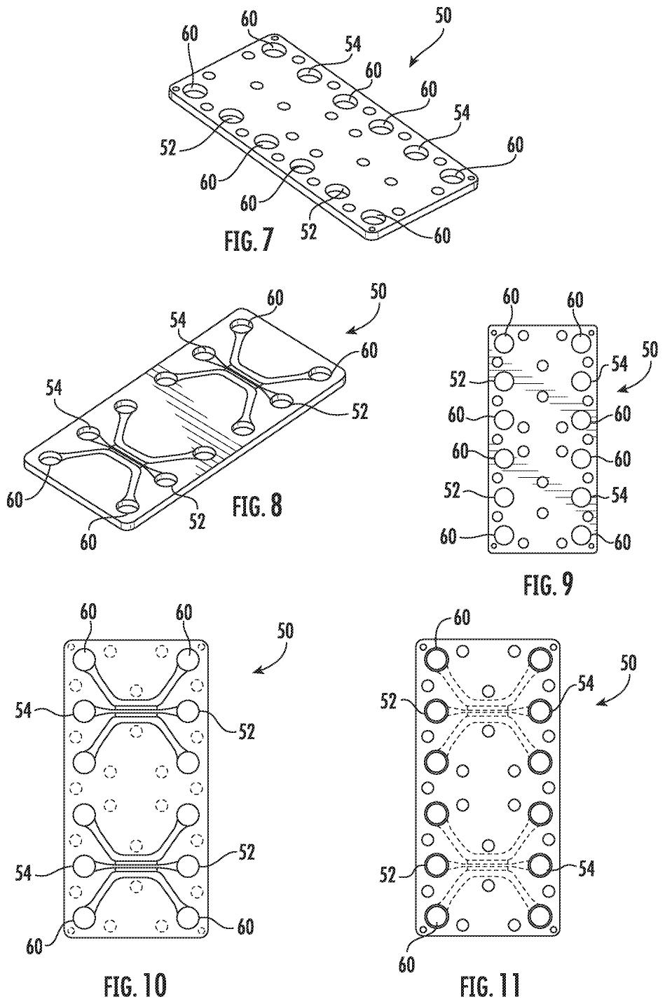

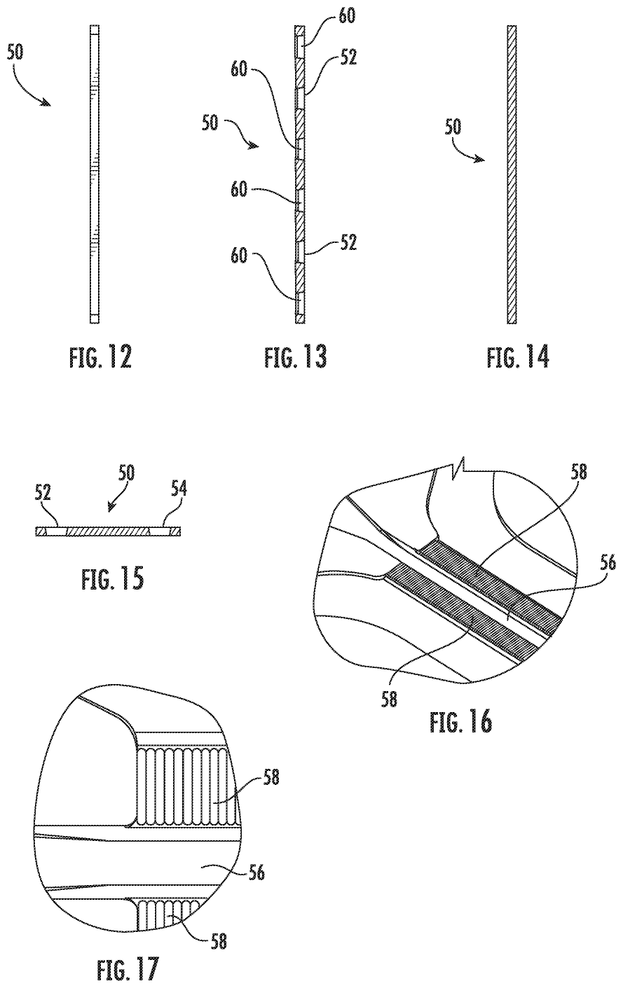

An embodiment of a microfluidic device is shown in FIGS. 7-17 and generally designated 50. The microfluidic device 50 has at least two compartments 52, 54 connected by a barrier region 56 having micron-sized grooves 58 at the bottom of a barrier region for maintaining fluidic isolation between the compartments 52, 54. The microfluidic device 50 can be applied to neuronal and non-neuronal cells. In the neuronal application, the grooves 58 are configured to direct the sites of neuronal attachment and the orientation of neurite outgrowth. Fluidic isolation of the compartments within the culture area provides the ability to deliver positive or negative stimuli to one compartment to expose only localized areas of the neurons, such as the soma, axons, or dendrites.

It is understood any number of chamber 52, 54 arrangements connected by a barrier region 56 are contemplated, including more than two independent chambers or compartments. In the embodiment shown, the microfabricated device 50 comprises a pair of separate culturing environments, or “chips”. In one embodiment, holes 60 may be placed in each device 50. The holes 60 are in fluid communication with the compartment 52, 54 and serve as loading inlets and cell medium reservoirs for nutrient and gas exchange. For example, each chip of the device 50 may contain four holes 60, two at either end of each compartment 52, 54.

It is understood that various sizes, shapes or geometries, and number of grooves are possible in the microfabricated device 50. Accordingly, the microfabricated device 50 is adaptable for use in a variety of culture environments.

The microfluidic device 50 may be created using microfabrication techniques, such as photolithography, using soft lithography techniques. The microfluidic culture devices may be fabricated from poly(dimethyl siloxane), PDMS. The fabrication processes and materials described herein are given for purposes of example only and other methods for making the microfluidic device described are also contemplated. Both glass and polystyrene tissue culture dishes can be used as substrates for the device. Use of an optically transparent polymer allows for live cell imaging.

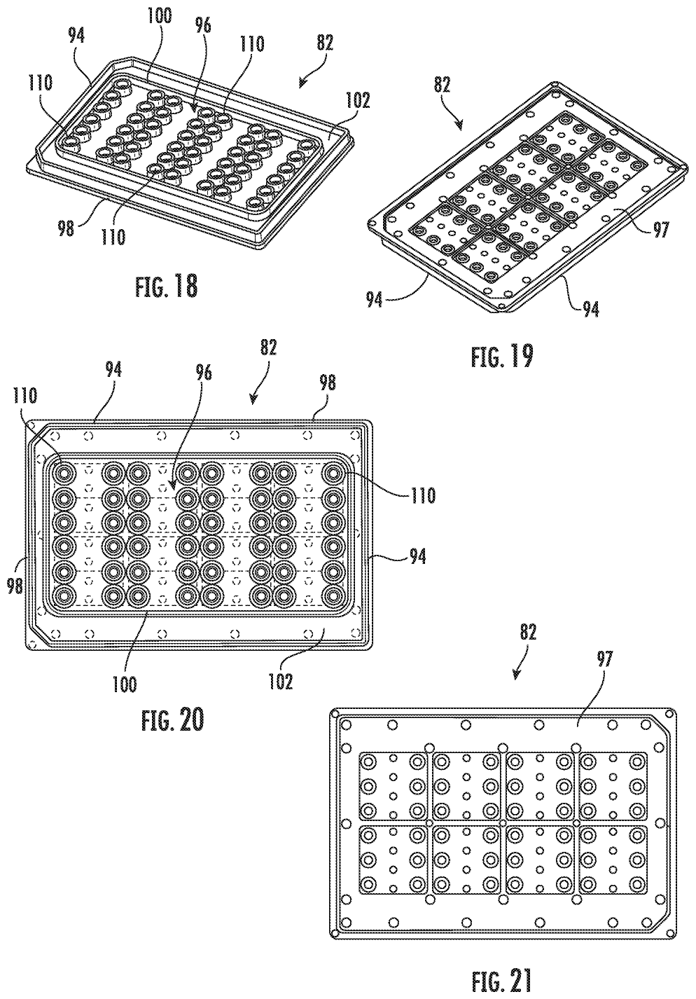

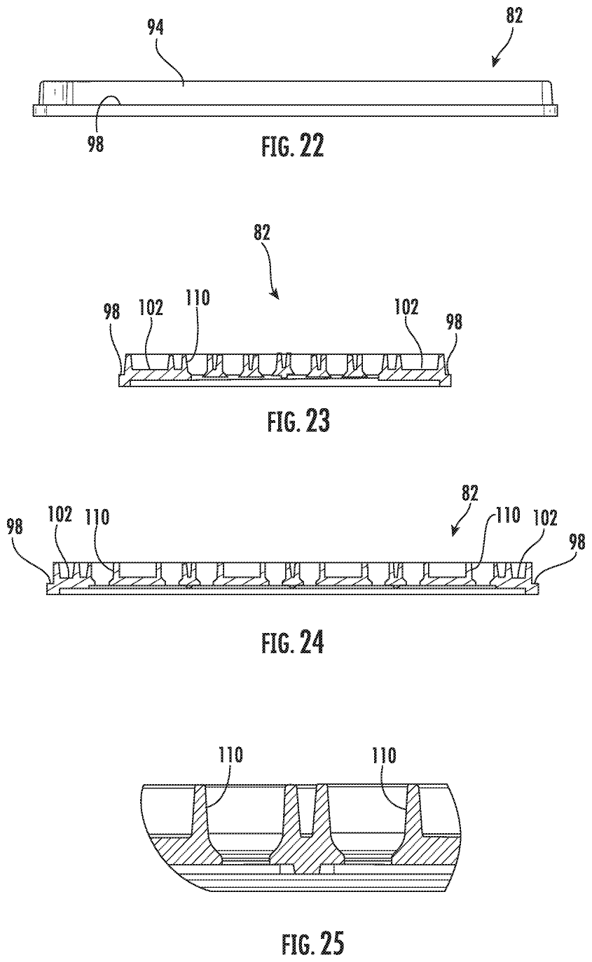

Another embodiment of an apparatus for humidifying cells during culturing for use with the microfluidic device 50 is shown in FIGS. 18-25. The humidifying apparatus comprises a tray assembly, generally designated at 80. The tray assembly 80 provides a closed environment for cell growth, as well as a means for easy loading of the microfluidic devices 50. In the embodiment shown, the tray assembly 80 has spaces for four microfabricated devices 50 as described herein. It is understood that the tray assembly 80 may be configured to hold more or less of the microfabricated devices 50.

The tray assembly 80 comprises a bottom tray 82 and a cover 84 (not shown). The bottom tray 82 is shown in the drawings as a generally rectangular member, although it is understood that the tray assembly 80 is not so limited and may have any convenient shape in addition to rectangular. The tray assembly 80 may be made by injection molding, CNC or other plastics manufacturing methods.

The tray assembly 80 may be formed from a number of suitable materials, including plastics or glass. A tray assembly 80 formed of plastic can be bendable and resilient to a certain extent, and thus insures that the bottom tray 82 and the cover can be readily assembled and separated from one another. In one embodiment, the tray assembly 80 is made of thermoformed, extruded or molded plastic resins. The tray assembly 80 may be substantially transparent or translucent for allowing observation of the interior of the tray assembly 80, including cellular material during growth. Alternatively, the tray assembly 80 may be opaque, such as black plastic, to prevent phototoxicity. It is understood that the tray assembly 80 is not intended to be limited by the materials listed here, but may be carried out using any material which allows the construction and operation of the humidifying apparatus described herein.

A continuous peripheral wall 94 extends orthogonally from an inner surface 96 of the bottom tray 82 and terminates in longitudinal edges. The peripheral wall 94 is spaced inwardly from the edges of the bottom tray 82 thereby forming an integral lip 98 extending along the edges of the bottom tray 82. A second continuous wall 100 extends orthogonally from the inner surface 96 of the bottom tray 82 and terminates in longitudinal edges the same distance from the inner surface 96 as the peripheral wall 94. The second wall 100 is spaced inwardly from the peripheral wall 94 thereby forming an open trough 102 between the walls 94, 100 and surrounding the microfabricated devices 50.

An outer surface 97 of the bottom tray 82 defines four parallel rectangular recesses 92. Each recess 92 is configured for receiving a microfabricated device 50. The portions of the outer surface 97 of the bottom tray 82 defining the recesses 92 include a continuous ledge 104 surrounding each chip. In one embodiment, the ledge 104 is configured to correspond to the size of each microfabricated device 50. A microfabricated device 50 is received in each of the recesses 92 and may be adhered within the outer surface 97 of the bottom tray 82.

The inner surface 96 of the bottom tray 82 further includes generally cylindrical reservoirs 110 opening through the bottom tray 82 and onto the microfabricated devices 50. In the embodiment shown, there is a reservoir 110 for each compartment 52, 54 and hole 60 in the microfabricated devices 50. The reservoirs 110 provide access for delivery of culture media or other fluids to the microfabricated devices 50.

Similar to the first embodiment, the cover includes a continuous peripheral wall extending orthogonally from the inner surface of the cover and terminating in longitudinal edges. The inner surface and the wall of the cover defines an open cavity for receiving the bottom tray 82 in an assembled condition. When assembled, the wall of the cover closely surrounds the peripheral wall 94 of the bottom tray 82 for a slip-fit connection between the cover and the bottom tray 82. In one embodiment, the distance of the terminal edge of the wall of the cover from the inner surface of the cover is greater than the distance of the terminal edge of the peripheral wall 94 of the bottom tray 82 from the inner surface 96 of the bottom tray 82. Accordingly, when the tray assembly 80 is in the assembled condition, the terminal edges of the wall of the cover engage the lip 98 at the edge of the bottom tray 82. This arrangement allows the user to predetermine the spacing between the inner surface 96 of the bottom tray 82 and the inner surface of the cover.

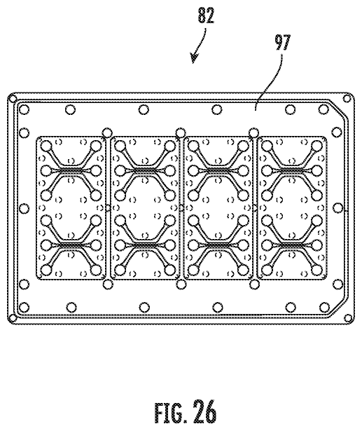

In use, cells are plated on the surfaces of the microfluidic devices 50. The microfluidic devices 50 are adhered in the recesses 92 of the outer surface 97 of the bottom tray 82 (FIG. 26). Four microfluidic devices 50 are shown in corresponding recesses 92. It is understood that the tray assembly 80 may be configured to accommodate more or less than four microfluidic devices 50 as desired. Water or other fluid is added to the trough 102 surrounding the microfluidic devices 50. A flange parallel to the inner surface 96 may extend from the peripheral wall 94 or the inner wall 100 partially over the trough 120 to minimize loss of fluid during handling of the tray assembly 80. The reservoirs 110 overlie the compartment 52, 54 and holes 60 defined by the microfluidic devices 50. The cover is then held over the bottom tray 82 so that the wall of the cover is adjacent the peripheral wall 94 of the bottom tray 82. The wall of the cover defines an area slightly larger than the area defined by the wall 94 of the bottom tray 82, which allows the cover to slide onto the bottom tray 82 with a minimum of manual urging. During assembly as the tray assembly 80 slides together, the terminal edges of the wall of the cover ultimately engage the lip 98 around the periphery of the bottom tray 82. With the tray assembly 80 in the assembled condition, the trough 102 is enclosed.

To disassemble the tray assembly 80, the cover is manually pulled away from the bottom tray 82 a sufficient distance to slidingly disengage the wall.

It is understood that although the embodiments of the tray assembly 80 shown herein depict a close-fitting walled arrangement, other arrangements for joining the cover and the bottom tray 82 are contemplated. For example, the cover may be pivotally mounted to the bottom tray 82 via a hinge assembly along one side for pivotal movement between a first open position and a second closed position.

Those of ordinary skill in the art will recognize that neurons are a test case and that the device described herein has applicability to other types of cells or biological type applications.

Although the apparatus and method for humidifying cell cultures has been shown and described in considerable detail with respect to only a few exemplary embodiments thereof, it should be understood by those skilled in the art that I do not intend to limit the humidifying apparatus to the embodiments since various modifications, omissions and additions may be made to the disclosed embodiments without materially departing from the novel teachings and advantages, particularly in light of the foregoing teachings. For example, the humidifying apparatus is suitable for use in a number of applications for culturing cells requiring a humid environment. Those of ordinary skill in the art, however, will recognize that neurons are just one example and that the device described herein has applicability to other types of cells or biological type applications. Accordingly, we intend to cover all such modifications, omission, additions and equivalents as may be included within the spirit and scope of the humidifying apparatus as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

I claim:

1. A device for use in culturing cells, the cell culturing device comprising:
a first body member having an inner surface defining a pair of spaced troughs opening to the inner surface for holding humidifying fluid, a portion of the inner surface between the troughs defining at least one recess having an opening;
a second body member having an inner surface; and
a substrate for cell growth,
wherein the substrate is configured to be received in the recess overlying the opening in the first body member, and
wherein the second body member is configured to be mounted to the first member such that the substrate is disposed between the inner surface of the first body member and the inner surface of the second body member.

2. A cell culturing device as recited in claim 1, wherein the first body member is elongated, and the troughs in the first body member are disposed at opposite ends of the first body member.

3. A cell culturing device as recited in claim 1, wherein the first body member and the second body member are transparent.

4. A cell culturing device as recited in claim 1, wherein the first body member and the second body member are opaque.

5. A cell culturing device as recited in 1, wherein the portion of the first body member defining the recess has a continuous ledge for receiving and supporting the substrate.

6. A cell culturing device as recited in claim 1, wherein each of the first body member and the second body member comprises
a base portion terminating in longitudinal edges, and
a continuous peripheral wall extending from the base portion and terminating in longitudinal edges,
wherein the base portion and peripheral wall of the second body member define an open cavity configured to receive the peripheral wall of the first body member for connecting the first body member and the second body member.

7. A cell culturing device as recited in claim 6, further comprising a flange extending along at least a portion of the longitudinal edges of the base portion of the first body member, and
wherein the terminal edges of the peripheral wall of the second body member contact the flange of the first body member when connecting for spacing the inner surface of the first body member from the inner surface of the second body member.

8. A cell culturing device as recited in claim 7, wherein the distance from the base portion to terminal edges of the wall of the first body member is less than the distance from the base portion to the terminal edges of the wall of the second body member.

9. A device for humidifying culturing cells growing on a substrate, the cell humidifying device comprising:
a first body member having an inner surface defining a pair of spaced troughs opening to the inner surface for holding a humidifying fluid, a portion of the inner surface between the troughs defining at least one recess having an opening for receiving the substrate; and
a second body member having an inner surface,
wherein the second body member is configured to be mounted to the first member such that the substrate is disposed between the inner surface of the first body member and the inner surface of the second body member.

10. A cell humidifying device as recited in claim 9, wherein the first body member is elongated, and the troughs in the first body member are disposed at opposite ends of the first body member.

11. A cell humidifying device as recited in claim 9, wherein the first body member and the second body member are transparent.

12. A cell humidifying device as recited in claim 9, wherein the first body member and the second body member are opaque.

13. A cell humidifying device as recited in 9, wherein the portion of the first body member defining the recess has a continuous ledge for receiving and supporting the substrate.

14. A cell humidifying device as recited in claim 9, wherein each of the first body member and the second body member comprises
a base portion terminating in longitudinal edges, and
a continuous peripheral wall extending from the base portion and terminating in longitudinal edges,
wherein the base portion and peripheral wall of the second body member define an open cavity configured to receive the peripheral wall of the first body member for connecting the first body member and the second body member.

15. A cell humidifying device as recited in claim 14, further comprising a flange extending along at least a portion of the longitudinal edges of the base portion of the first body member, and
wherein the terminal edges of the peripheral wall of the second body member contact the flange of the first body member when connecting for spacing the inner surface of the first body member from the inner surface of the second body member.

16. A cell humidifying device as recited in claim 15, wherein the distance from the base portion to terminal edges of the wall of the first body member is less than the distance from the base portion to the terminal edges of the wall of the second body member.

17. A device for use in culturing cells, the cell culturing device comprising:
a first body member having an inner surface and an outer surface, the inner surface defining a peripheral trough opening to the inner surface for holding humidifying fluid, the outer surface defining at least one recess within the area defined by the trough, and having at least one opening between the inner surface and the recess;
a second body member having an inner surface; and
a substrate having a surface for cell growth,
wherein the substrate is configured to be received in the recess overlying the at least one opening in the first body member such that the surface for cell growth is disposed adjacent to the outer surface of the first body member, and
wherein the second body member is configured to be mounted to the first member.

18. A cell culturing device as recited in claim 17, wherein the first body member is elongated, and wherein the trough is continuous around the periphery of the first body member.

19. A cell culturing device as recited in claim 17, wherein the first body member and the second body member are transparent.

20. A cell culturing device as recited in claim 17, wherein the first body member and the second body member are opaque.

21. A cell culturing device as recited in 17, wherein the portion of the first body member defining the recess has a continuous ledge for receiving and supporting the substrate.

22. A cell culturing device as recited in claim 17, wherein each of the first body member and the second body member comprises
a base portion terminating in longitudinal edges, and
a continuous peripheral wall extending from the base portion and terminating in longitudinal edges,
wherein the base portion and peripheral wall of the second body member define an open cavity configured to receive the peripheral wall of the first body member for connecting the first body member and the second body member.

23. A cell culturing device as recited in claim 22, further comprising a flange extending along at least a portion of the longitudinal edges of the base portion of the first body member, and
wherein the terminal edges of the peripheral wall of the second body member contact the flange of the first body member when connecting for spacing the inner surface of the first body member from the inner surface of the second body member.

24. A cell culturing device as recited in claim 23, wherein the distance from the base portion to terminal edges of the wall of the first body member is less than the distance from the base portion to the terminal edges of the wall of the second body member.
